SEQUENCE LISTING (23 sequences)

SEQ ID NO: 1 - protein, 60 aa, Artificial Sequence
  Lipid binding domain
WXXDXXXXXCXXCXXXXXXXXXXHHCRXCXXXXCXXCXXXXXXXXXXXXXXXXRVCXXCX

SEQ ID NO: 2 - protein, 66 aa, Homo sapiens
LNRKWAEDNEVQNCMACGKGFSVTVRRHHCRQCGNIFCAECSAKNALTPSSKKPVRVCDACFNDLQ

SEQ ID NO: 3 - protein, 65 aa, Mus musculus
RAPDWVDAEECHRCRVQFGVVTRKHHCRACGQIFCGKCSSKYSTIPKFGIEKEVRVCEPCYEQLN

SEQ ID NO: 4 - protein, 67 aa, Homo sapiens
VAPVWVPDSQAPNCMKCEARFTFTKRRHHCRACGKVFCASCCSLKCKLLYMDRKEARVCVICHSVLM

SEQ ID NO: 5 - protein, 64 aa, Homo sapiens
SVVPWVNDQDVPFCPDCGNKFSIRNRRHHCRLCGSIMCKKCMELISLPDDDRIRCCTHCKDTLL

SEQ ID NO: 6 - protein, 68 aa, Rattus norvegicus
RAPRWIRDNEVTMCMKCKESFNALTRRRHHCRACGHVVCWKCSDYKAQLEYDGGRLNKVCKDCYQIMS

SEQ ID NO: 7 - protein, 68 aa, Homo sapiens
RAPTPIREKEVTMCMRCQEPFNSITKRRHHCKACGHVVCGKCSEFRARLVYDNNRSNRVCTDCYVALH

SEQ ID NO: 8 - protein, 14 aa, Homo sapiens
CEARFTFTKRRHHS

SEQ ID NO: 9 - protein, 14 aa, Homo sapiens
CEARFSVTVRRHHC

SEQ ID NO: 10 - protein, 59 aa, Homo sapiens
WVPDSQAPNCMKCEARFTFTKRRHHCRACGKVFCASCCSLKCKLLYMDRKEARVCVICH

SEQ ID NO: 11 - protein, 4 aa, Artificial Sequence
  Motif
WXXD

SEQ ID NO: 12 - protein, 6 aa, Artificial Sequence
  Motif
XXHHCR

SEQ ID NO: 13 - protein, 4 aa, Artificial Sequence
  Motif
XXXX

SEQ ID NO: 14 - protein, 4 aa, Artificial Sequence
  Example of useful Turret loop
AFFR

SEQ ID NO: 15 - protein, 4 aa, Artificial Sequence
  Example of useful Turret loop
AFIR

SEQ ID NO: 16 - protein, 4 aa, Artificial Sequence
  Example of useful Turret loop
AIFR

SEQ ID NO: 17 - protein, 4 aa, Artificial Sequence
  Example of useful Turret loop
AFFK

SEQ ID NO: 18 - protein, 4 aa, Artificial Sequence
  Example of useful Turret loop
AFIK

SEQ ID NO: 19 - protein, 4 aa, Artificial Sequence
  Example of useful Turret loop
AIFK

SEQ ID NO: 20 - protein, 4 aa, Artificial Sequence
  Example of useful Turret loop
TFTK

SEQ ID NO: 21 - protein, 5 aa, Artificial Sequence
  Specific example
GLSAL

SEQ ID NO: 22 - protein, 54 aa, Homo sapiens
WAEDNEVQNCMACGKGFSVTVRRHHCRQCGNIFCAECSAKNALTPSSKKPVRVC

SEQ ID NO: 23 - protein, 55 aa, Homo sapiens
WVPDSQAPNCMKCEARFTFTKRRHHCRACGKVFCASCCSLKCKLLYMDRKEARVC

(19) United States
(12) Patent Application Publication
(10) Pub. No.: US 2004/0192601 A1
(43) Pub. Date: Sep. 30, 2004

(54) Lipid binding molecules and methods of use

(76) Inventors: Silvia Corvera, Westborough, MA (US); David Lambright, Marlboro, MA (US)

Correspondence Address: FISH & RICHARDSON PC, 225 FRANKLIN ST, BOSTON, MA, 02110, US

(21) Appl. No.: 10/634,679

(22) Filed: Aug. 4, 2003

Related U.S. Application Data
(60) Provisional application No. 60/400,619, filed on Aug. 2, 2002.

(51) Int. Cl. A61K 49/00; G01N 33/53; C07K 14/775

(52) U.S. Cl. 514/12; 424/9.6; 530/324; 435/7.2

(57) ABSTRACT

The invention relates to new lipid binding molecules that include a lipid binding domain and a reporter group. The new molecules can be used, for example, to detect and visualize lipids, such as phosphatidylinositol 3-phosphate.

### CROSS-REFERENCE TO RELATED APPLICATIONS

This application claims priority from U.S. Provisional Patent Application Serial No. 60/400,619, filed on Aug. 2, 2002, the contents of which is incorporated herein by reference in its entirety.

### GOVERNMENT FUNDING

This invention was made from Government support under grant number 1PO1 DK60564-01 awarded by the NIH and NIDDK. The Government has certain rights in the invention.

### FIELD OF THE INVENTION

The invention relates to lipid binding molecules and methods of use.

### BACKGROUND

The detection of lipids has allowed the discovery of many important cellular processes. Lipids can be detected by proteins that bind to specific portions of lipid molecules. One protein domain known to bind to lipids is the so-called FYVE domain, which is named after the first four proteins (Fab1, YOTB, Vac1, and EEA1) that were found to contain this motif. The FYVE domain is an ˜80-amino acid sequence, and contains several conserved regions that allow this and related domains to bind to lipids. The FYVE domain was defined by Stenmark and co-workers, who identified it as part of the sequence of an endosomal protein, EEA1, and recognized it as a specific subclass of the RING (a zinc-finger) motif (Stenmark et al. (1996) J. Biol. Chem., 271:24048-24054, Simonsen et al. (1998) Nature, 394:494-498).

The interaction of the FYVE domain with phosphatidylinositol 3-phosphate (PI3P) was elucidated from two findings. First, the interaction of EEA1 with membranes was sensitive to the phosphatidylinositol (PI3-kinase (PI3K) inhibitor, wortmannin (Patki et al., (1997) Proc. Natl. Acad. Sci. U.S.A., 94:7326-7330); and PI3K is the enzyme that catalyzes the phosphorylation of PI at the 3 position of the inositol headgroup to form PI3P. FIG. 1 shows the structure of PI. The PI3K inhibitor prevented the formation of PI3P and in its absence EEA1 could not bind to the membranes. Second, the EEA1 FYVE domain was found to have a high affinity for PI3P (Burd et al., (1998) Mol. Cell, 2:157-162, Gaullier et al., (1998) Nature, 394:432-433, Patki et al., (1998) Nature, 394:433-434).

The FYVE domain has been found in many other known cellular proteins, e.g., SARA, HRS, FRABIN, Rabenosyn, FGD1, and DFCP1, many of which appear to have roles in signaling or membrane trafficking, or both (Komada et al. (1999) Genes. Dev., 13:1475-1485, Mills et al. (1998) Curr. Biol., 8:881-884, Obaishi et al. (1998) J. Biol. Chem., 273:18697-18700, Otto, et al. (2000) Curr. Biol., 10:345-348, Shisheva et al., (1999) Mol. Cell. Biol., 19:623-634, Tsukazaki et al. (1998) Cell, 95:779-791, Zhao et al. (2000) Biochem. Biophys. Res. Commun., 270:222-229, Zheng et al. (1996) J. Biol. Chem., 271:33169-33172).

### SUMMARY

The invention is based, in part, on the discovery of specific polypeptide domains and motifs that bind to lipids such as phosphatidylinositol 3-phosphate (PI3P) with high specificity and affinity. Lipid binding molecules that include these domains and/or motifs can be used to detect lipids with very high specificity and affinity, e.g., in screening assays.

In one aspect, the invention features a lipid binding domain having the sequence

(SEQ ID NO:1)

WxxDxxxxxCxxCxxxx(A/T)uuj(R/K)(R/K)HHCR(A/G/V)Cxxx
xCxxCxxxxxxxxxxuuuxuuRVCxxCx,

wherein x is any amino acid; u is a highly hydrophobic residue such as F, V, I, L, M, W, Y, or T (or selected from any subgroup thereof); and j is a positively charged residue such as R or K. In certain embodiments, the “uuuxuu” sequence upstream of RVC in the lipid binding domain (SEQ ID NO:1) can be xxxxuu. This lipid binding domain can be linked to a reporter group to form a lipid binding molecule.

In these lipid binding molecules, the lipid binding domain can bind to PI3P, e.g., in a cell, or on an endosome or liposome. For example, the lipid binding molecule can bind to PI3P in a solid phase binding assay, or in a protein overlay assay. In these assays, the reporter group can attach or bind to a substrate, or provide a visible marker. In certain embodiments, the reporter group can be one member of a binding pair, and the substrate can be a second member of the binding pair. For example, the reporter group can be selected from the group of glutathione S-transferase (GST), 6His, FLAG, Green Fluorescent Protein (GFP), chitin binding protein, cellulase, maltose binding protein, dihydrofolate reductases, FK506 Binding Protein (FKBP), FKBPF36V, an antibody, a fluorescently labeled antibody, and an antibody fragment. In other embodiments, the reporter group can be a fluorescent moiety, such as GFP. The reporter group can also be a labeled antibody.

In another aspect, the invention features an assay for detecting a lipid, e.g., PI3P, for example within a lipid bilayer in a sample, by a) obtaining a new lipid binding molecule; b) mixing the lipid binding molecule with the sample under conditions that enable the lipid binding molecule to bind to a lipid in the sample to form a complex; and c) detecting the complex as an indication of the presence of the lipid in the sample.

In any of these embodiments, the lipid binding domain can be the same as, or essentially the same as, a FYVE domain of a SARA protein, and can include naturally occurring and/or synthetic amino acids. The lipid binding domain can be a polypeptide of about 30, 40, 50, 55, 56, 57, 58, 59, or 60 amino acids in length, and the reporter group can be glutathione S-transferase. The lipid binding domain can include a so-called “Turret Loop” selected from the group: AFFR (SEQ ID NO:14), AFIR (SEQ ID NO:15), AIFR (SEQ ID NO:16), AFFK (SEQ ID NO:17), AFIK (SEQ ID NO:18), AIFK (SEQ ID NO:19), and TFTK (SEQ ID NO:20).

In another aspect, the invention features a method of locating a lipid-containing cellular organelle, e.g., containing PI3P, within a cell by a) obtaining a composition including a lipid binding molecule; b) applying the composition to the cell under conditions that enable the lipid binding molecule to enter the cell; and c) detecting the reporter group of the lipid binding molecule, whereby the location of the reporter group within the cell indicates the location of the cellular organelle. This method can be performed in vivo or in vitro, and the cellular organelle can be an endosome, or an endosome that includes a phagosome, or a liposome. In certain embodiments, the reporter group can provide a visual signal and the method can further include visualizing the location of the cellular organelle.

The invention also features a method of diagnosing a subject for infection by a microorganism, by a) obtaining a cell from the subject; b) binding a lipid binding molecule to a phagosome in the cell; c) visualizing the reporter group on a phagosome; and d) determining whether the phagosome is capable of fusing to a lysosome, wherein a phagosome that cannot fuse to a lysosome indicates that the cell is infected by a microorganism. In this method, the microorganism can be Mycobacterium tuberculosis, and the cell can be derived from a subject at risk for infection by the microorganism. The subject can be an animal, e.g., a mammal (such as a dog, cat, cow, pig, horse, or a non-human primate), or a human.

In another aspect, the invention includes a method of determining whether a test compound, such as an antibiotic, is a candidate compound for treating Mycobacterium tuberculosis by a) binding a lipid binding molecule to a phagosome in a cell infected by M. tuberculosis; b) applying a test compound to the infected cell; and c) visualizing the phagosome before and after application of the test compound; wherein a phagosome that is capable of fusing to a lysosome after application of the test compound indicates that the test compound is a candidate compound to treat M. tuberculosis. A “candidate” compound is a compound or composition that can be used as is to treat a particular disorder, or that can be modified, e.g., derivatized, using standard techniques to form a compound or composition that can be used to treat a disorder.

A “lipid binding domain” is an isolated, naturally occurring, or artificially-produced polypeptide, peptidomimetic, or peptoid molecule that binds with high affinity to a lipid, e.g., phosphatidylinositol 3-phosphate. Under physiological conditions, the lipid binding domain can bind to a lipid found in a lipid bilayer, e.g., a lipid on a cellular organelle, e.g., an endosome.

A “reporter group” is a molecule that can either (i) bind to a substrate, e.g., a ligand on a substrate, thereby allowing molecules containing the reporter group to be separated and isolated from a mixture (e.g., glutathione-S-transferase, and glutathione (see, e.g., U.S. Pat. No. 5,654,176); hexa-histidine and Ni²⁺ (see, e.g., German Patent No. DE 19507 166); chitin binding protein and chitin; cellulase (CBD) and cellulose; maltose binding protein and amylose, or maltose; dihydrofolate reductases and methotrexate; FKBP and FK506, and an antibody and a peptide epitope (see, e.g., Kolodziej and Young (1991) Methods Enz. 194:508-519 for general methods of providing an epitope tag); or (ii) provide a measurable marker or label, thereby allowing molecules containing the reporter group to be detected and monitored, e.g., green fluorescent protein (GFP) or other luminescent proteins.

A “lipid binding molecule” is formed by attaching or linking, e.g., fusing, a lipid binding domain to a reporter group using covalent or non-covalent bonds. The lipid binding domain and the reporter group should be linked at a distance such that the reporter group does not interfere with the lipid binding domain binding to a lipid, e.g., phosphatidylinositol 3-phosphate. Thus, a molecular spacer, e.g., a peptide, polyethylene glycol-based spaced linker, or disulfide bond can be inserted between the lipid binding domain and the reporter group.

A “peptoid” is an oligomer of N-substituted glycines. A peptoid can be synthesized from a variety of different N-alkylglycines that have side chains similar to amino acid side chains, e.g., as described in Simon et al., (1992) PNAS 89:9367-9371. It can serve as a motif for the generation of chemically diverse libraries of novel molecules. As an alternative to natural polymers, it is a modular system that allows one to synthesize monomers in large numbers. The monomers have a wide variety of functional groups presented as side chains on an oligomeric backbone, the linking chemistry is high yielding and amenable to automation. The linkage in a peptoid is resistant to hydrolytic enzymes such as proteases. Another advantage is that the monomers are achiral.

A “peptidomimetic” is a chemical variant of a polypeptide (e.g., a peptide) such as a peptoid in which the side chains of the polypeptide are substantially maintained in the variant, yet the chemical backbone of the peptidomimetic is altered relative to the polypeptide in one or more peptide bonds.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The lipid binding molecules described herein have several advantages. In comparison experiments of the known FYVE domain-containing proteins, the lipid binding domain of the new molecules has shown maximal and efficient lipid bilayer binding. In addition, the new lipid binding molecules can be used in solid phase binding assays and protein overlay experiments.

Other features and advantages of the invention will be apparent from the following detailed description, and from the claims.

### BRIEF DESCRIPTION OF THE DRAWINGS

FIG. 1 is the chemical structure of phosphatidylinositol (PI). The inositol headgroup is denoted and the positions on the ring are numbered.

FIGS. 2A-2D are microscopy images of live cells that have been transfected with GFP-tagged FYVE domains of the EEA1 and the SARA proteins. FIG. 2A is a microscopic image of a cell expressing the FYVE domain of EEA1 fused to GFP. FIG. 2B is a microscopic image of a cell expressing the FYVE domain of FRABIN fused to GFP. FIG. 2C is a microscopic image of a cell expressing the FYVE domain of Hrs fused to GFP. FIG. 2D is a microscopic image of a cell expressing the FYVE domain of SARA fused to GFP.

FIG. 3 is an amino acid sequence alignment of the FYVE domains of six different proteins (SEQ ID NOs:2-7). The conserved and non-conserved motifs are noted. The Lipid Binding Domain (SEQ ID NO:1) is also shown.

FIGS. 4A-4B are microscopy images of cells transfected with either GFP-tagged FYVE domain of SARA (CEARFTFTKRRHHS; SEQ ID NO:8, bold sequence is the Turret Loop) or GFP-tagged FYVE domain of SARA containing the turret loop from EEA1 protein (CEARFSVTVRRHHC; SEQ ID NO:9, bold sequence is the Turret Loop). FIG. 4A is microscopic image of a cell expressing the FYVE domain of SARA fused to GFP. FIG. 4B is a microscopic image of a cell expressing the FYVE domain of SARA containing the Turret loop of EEA1 fused to GFP.

FIGS. 5A-5C are EEA1 FYVE domain protein structure drawings modeled from x-ray crystallographic images. FIG. 5A is a polypeptide backbone trace superimposed on a surface rendering of the EEA1 FYVE domain. FIG. 5B is a ribbon drawing of specifically the FYVE domain core of EEA1 without the coiled coil region. FIG. 5C is a surface rendering of the EEA1 FYVE domain core without the coiled coil region.

FIGS. 6A-6C are schematic illustrations of the components of an experiment using chemical dimerization. FIG. 6A shows the FYVE domain construct used in the experiment. FIG. 6B shows the chemical structure of the chemical crosslinker used to dimerize FYVE domain monomers. FIG. 6C shows how the experiment proceeds at the molecular level.

FIG. 7 is a chart of sixteen microscope images of live cells in which the isolated FYVE domains of four different proteins (EEA1, Hrs, Frabin, and SARA) are exposed to the dimerizer AP20187 (FIG. 6B) for different lengths of time (0, 5, 10, and 15 minutes) and then applied to the cells. The longer the protein is exposed to the dimerizer, the greater the endosome binding (up to about 40 minutes, when all protein is bound.

FIG. 8 is a representation of the results of a solid phase binding assay in the presence and absence of liposomes and inhibitors.

FIG. 9 is a representation of the results of a solid phase binding assay in the presence and absence of inhibitors done in two separate experiments.

FIGS. 10A and 10B are a series of microscope images of live cells in the form of a chart. In FIG. 10A, the chart shows images that resulted when cells were exposed to four different lipid binding molecules (EEA1 with combinations of a turret loop from itself (TLe) or from SARA (TLs) and/or a dimer interface loop from itself (DIe) or from SARA (DIs), in the absence (top row) or presence (bottom row) of the dimerizer AP20187. FIG. 10B is a similar chart showing images that resulted when cells were exposed to two different lipid binding molecules (SARA with combinations of a turret loop from itself (TLs) or from EEA 1 (TLe) and a dimer interface loop from itself (DIs), in the absence (top row) or presence (bottom row) of the dimerizer AP20187.

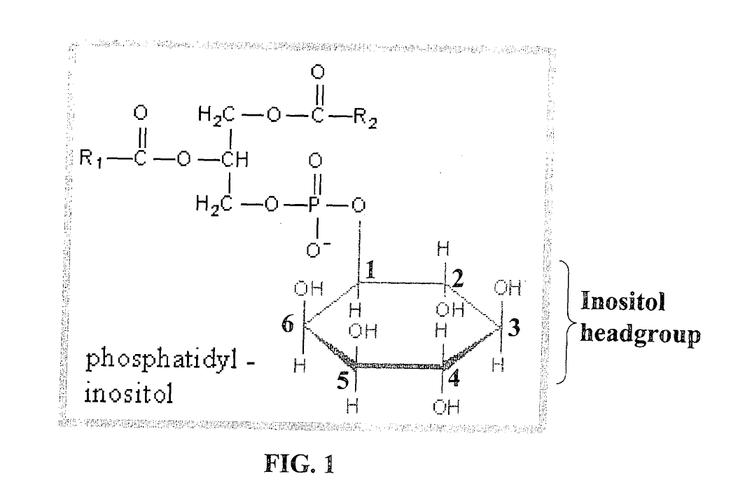

FIG. 1C is a representation of a sequence comparison between portions of EEA1 (SEQ ID NO:22) and SARA (SEQ ID NO:23), showing the turret loops (TL) and dimer interface loops (DI).

FIGS. 11A and 11B are a pair of charts of microscope images showing the effect of altering the turret loop on endosomal localization of FYVE domain of EEA1 (FIG. 11A) or SARA (FIG. 11B), in the absence (top row) and presence (bottom row) of the dimerizer AP20187.

### DETAILED DESCRIPTION

The invention is based, in part, on the discovery that the FYVE domain of the SARA protein binds to PI3P with higher affinity and efficiency than the FYVE domains of other proteins, even in the context of a lipid bilayer. Moreover, the non-conserved residues in the FYVE domains are important for their biological function. For example, motifs referred to herein as the “turret loop” and the “dimer interface loop” determine the ability of any given FYVE domain to effectively bind to PIP3 in a membrane bilayers, such as in a liposome or endosome. Based on these finding, the invention provides new lipid binding molecules, e.g., synthetic molecules, that include new, e.g., artificial, lipid binding domains that include key motifs that, in turn, provide the lipid binding molecules with the ability to bind to lipids with high specificity and affinity. The new lipid binding molecules can be used in novel assays to detect the presence and levels of lipids such as PI3P.

In particular, methods described herein take advantage of the finding that the FYVE domain of a particular protein, the SARA protein, binds to PI3P both in cell lysates and in whole cells (Example 1, infra) with greater affinity, avidity, and efficiency than any other currently known FYVE domain-containing protein. The sequence of the FYVE domain in SARA was used to create new, artificially produced lipid binding domains that have high affinity for lipids, and that can be tailored for specific research needs.

Lipid Binding Molecules

The new lipid binding molecules bind to lipids such as PI3P, which is produced from the phosphorylation of phosphatidylinositol on the 3 position of the inositol ring by the enzyme PI3K. FIG. 1 is an illustration of phosphatidylinositol with the inositol ring numbered. PI3K is an important enzyme present in all cells and is involved in numerous basic cellular processes, including cell survival, cell division, cell motility, membrane trafficking and the establishment of cell architecture, as well as more specific cell processes including the regulation of phagocytosis and phagosome maturation, essential for the elimination of invading microorganisms.

Important in studying this enzyme is the detection of its products. PI3P in particular is evaluated and monitored in the study of endosome formation, maturation, function, and fusion. Thus, a lipid-binding molecule as described herein, which specifically recognizes and can isolate PI3P from experimental lysates, as well as specifically recognizes PI3P and allows for its visualization within the cell, has many experimental, diagnostic, and therapeutic uses.

The new lipid binding molecules include two main portions, a lipid binding domain and a reporter group linked together in such a way as not to interfere with the ability of the lipid binding domain to bind to a lipid, e.g., within a lipid bilayer within a cell. The lipid binding domain and the reporter group can be linked directly or via a spacer molecule.

Lipid Binding Domains

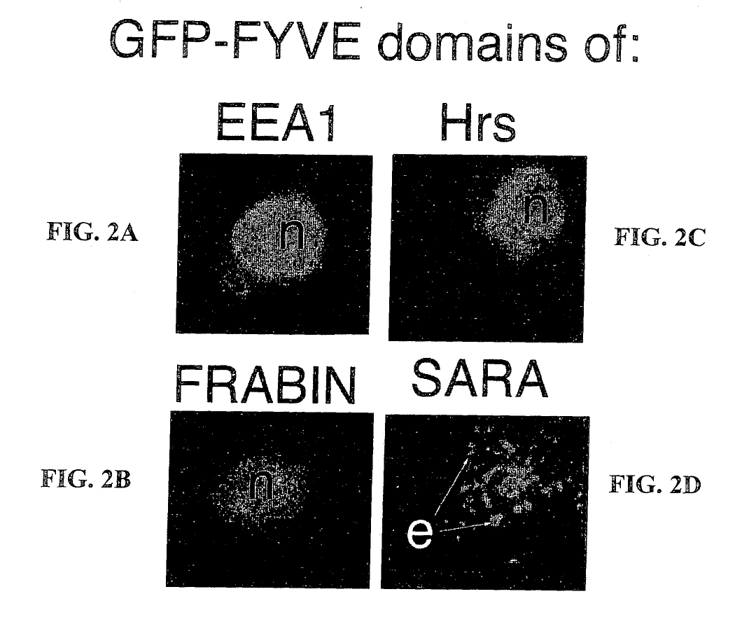

The lipid binding molecules described herein have key features in their lipid binding domains for binding to PI3P with high affinity in lysate binding and to lipids such as PI3P on endosomes within cells. Described below are the conserved residues in these domains that bind to the inositol head group and non-conserved residues that allow in vivo binding, and thus visualization of endosomes. Experiments also show that the ability of the FYVE domain to dimerize is important for this lipid binding domain to bind to endosome-bound PI3P. The FYVE domain of the SARA protein is used herein as it contains these key features and has been found to be the best endosome binder in a comparison of all the known FYVE domain-containing proteins (see FIG. 2, “n” refers to nucleus and “e” refers to endosome). As will be described in further detail below, both the isolated, naturally occurring form of the SARA FYVE domain and synthetic FYVE domains with the key motifs described herein can be used as the lipid binding domain.

The FYVE domain of SARA has the amino acid sequence:

| | |
|---|---|
| WVPDSQAPNCMKCEARFTFTKRRHHCRACGKVFCAS | (SEQ ID NO:10) |
| CCSLKCKLLYMDRKEARVCVICH,. | |

Based on this naturally occurring sequence, the new lipid binding domain includes or has the sequence:

| | |
|---|---|
| WxxDxxxxxCxxCxxxx(A/T)uuj(R/K)(R/K)HH | (SEQ ID NO:1) |
| CR(A/G/V)CxxxxCxxCxxxxxxxxxxuuuxuuRVC | |
| xxCx, | |

wherein, x=any amino acid; u=a highly hydrophobic residue such as F, V, I, L, M, or W; and j is a positively charged residue such as R or K. In some embodiments, the sequence “uuuxuu” upstream of the RVC motif can be “xxxxuu.”

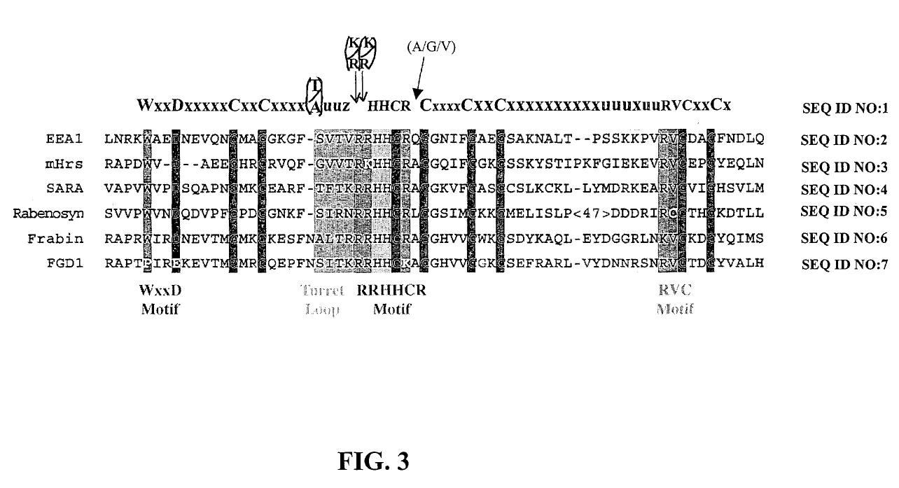

The lipid binding domain of the new lipid binding molecules has certain conserved motifs. These motifs allow it to bind to the inositol head group of phosphoinositide, and include: the “WxxD motif” (x=any amino acids) (SEQ ID NO:11), the “(R/K)(R/K)HHCR motif” (R or K in the first and second position) (SEQ ID NO:12) and the “RVC motif.” These are designated in FIG. 3. Experiments removing or mutating these conserved residues show that binding to PI3P is inhibited or reduced, thus revealing their importance in this interaction. These motifs can interact with each other to allow specific binding. For example, the W residue, which does not interact directly with the inositol headgroup, is found located proximal to the R residues three-dimensionally, and serves to position these residues for optimal binding to the inositol head group. In addition, two cysteine residues must be located such that disulfide bonds can form the correct three-dimensional structure of the domain. These conserved motifs and cysteine residues are therefore important for the binding specificity of the lipid binding domain to PI3P.

The known FYVE domains also contain non-conserved regions, which explain their differential affinities for PI3P, and thus should be useful to provide varied functions. These are also reflected in the new lipid binding domain. Located immediately upstream of the (R/K)(R/K)HHCR motif (SEQ ID NO:12) are four important hydrophobic residues referred to as the “Turret loop” ((A/T/S)uuj) (SEQ ID NO:13). This loop domain does not actually contribute to the inositol head group interaction, but is found to be critical for binding to PI3P within a physiological context. These residues are important in contacting the lipid bilayer found in cellular organelles, e.g., endosomes. Their hydrophobicity and positive charge enable stronger interaction with the lipid bilayer. Examples of useful Turret loops include AFFR (SEQ ID NO:14), AFIR (SEQ ID NO:15), AIFR (SEQ ID NO:16), AFFK (SEQ ID NO:17), AFIK (SEQ ID NO:18), AIFK (SEQ ID NO:19), and TFTK (SEQ ID NO:20).

Also important in the interaction with the lipid bilayer are the two to six residues preceding the RVC motif. Five of these six, and especially the two residues closest to the RVC motif, are ideally highly hydrophobic to serve this purpose. Examples of these pairs of residues include VI, IV, LF, VF, and IF.

Another non-conserved residue is the A residue immediately after the (R/K)(R/K)HHCR motif (SEQ ID NO:12), in the SARA FYVE domain. In making a new lipid binding molecule, the lipid binding domain ideally has a small, moderately hydrophobic, small residue at this position, e.g., A, G, or V, which provide characteristics important in allowing spontaneous dimerization of lipid binding domain monomers. Methods of artificially dimerizing the new molecules are described below.

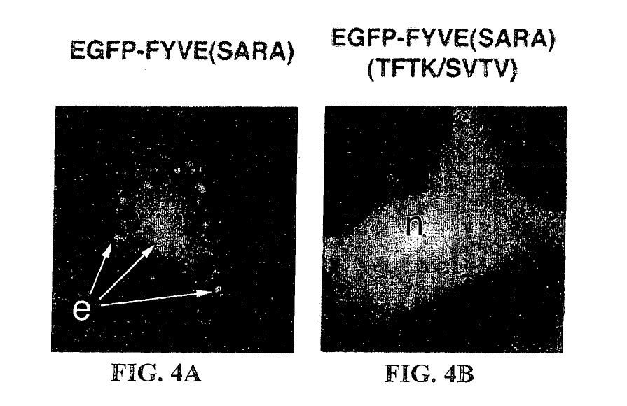

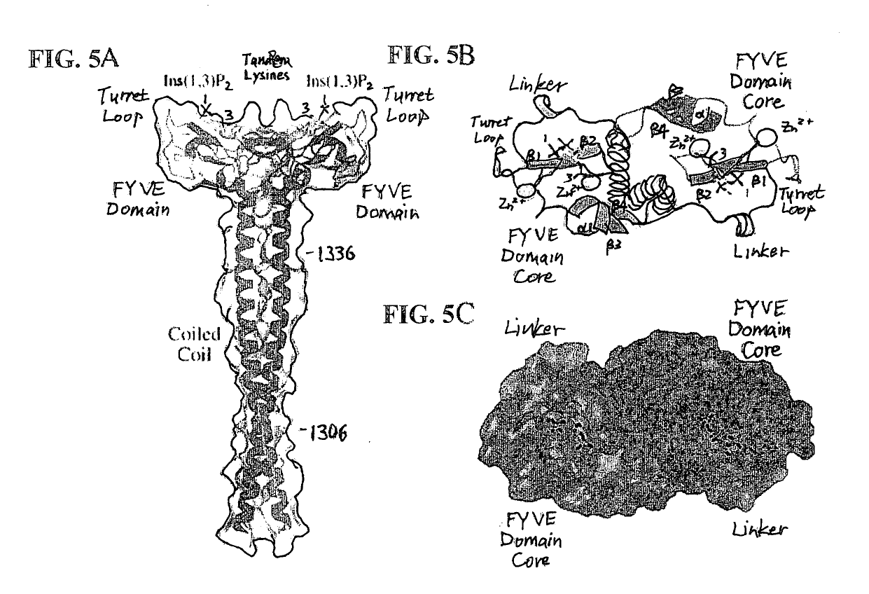

The non-conserved residues of the Turret loop have been experimentally tested in a comparison between the FYVE domain of SARA and the FYVE domain of SARA containing the Turret loop of EEA1. EEA1 is a protein containing identical conserved residues, but varies from SARA in the non-conserved Turret loop. The presence of the EEA1 Turret loop prevented the modified SARA FYVE from localizing to endosomes in a cellular context. This result is illustrated in FIGS. 4A and 4B (“e” refers to endosome and “n” refers to nucleus). In FIG. 4A, punctate staining of the FYVE domain of SARA, as visualized by green fluorescent protein (GFP) as the reporter group, is evidence of localization of the lipid binding molecule to the endosome. As can be seen in FIG. 4B, however, a lipid binding molecule consisting of the SARA FYVE domain that has the EEA1 Turret loop does not localize to the endosome, as indicated by uniform staining of the nucleus. The crystal structure of EEA1 reveals that the turret loop is positioned in a way that can interact with the lipid membrane surface (FIG. 5). This may explain why the Turret loop of SARA, with its more highly hydrophobic residues, has an enhanced binding to PI3P present in endosomes. The crystal structure also reveals an extensive interface formed between two monomers of the FYVE domain. This interface, or dimerization site could also contribute to improved endosome binding.

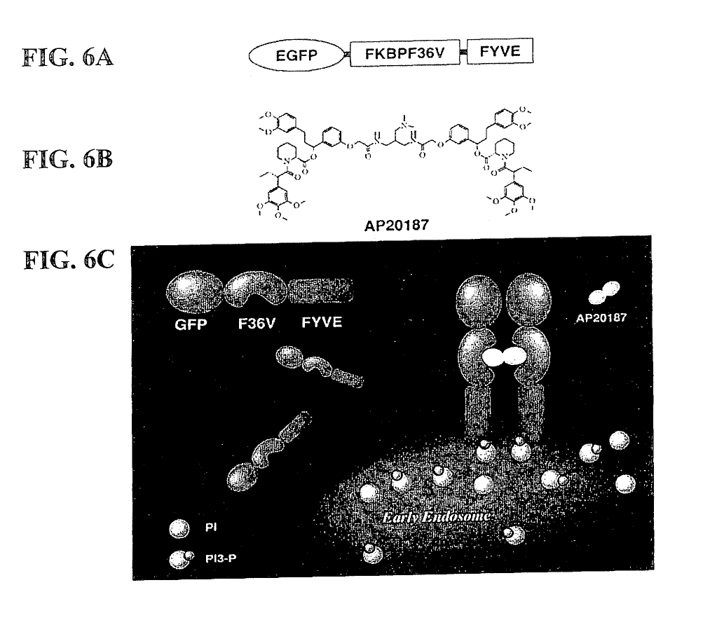

Dimerization of the FYVE domain may also play an important role in the recognition of intracellular PI3P. To show the importance of dimerization, FYVE domains were artificially dimerized. FIGS. 6A-C provide a schematic diagram of the dimerization process intended in this experiment. As shown pictorially in FIG. 6A, a mutant of FKBP (referred to in the figure as FKBF36V) that binds with high affinity to the chemical dimerizer, AP20187 (shown in FIG. 6B), was incorporated into the FYVE domains of different proteins, EEA1, Hrs, FRABIN, and SARA, fused to the recognition domain, GFP. FIG. 6C shows a schematic illustration of the formation of dimers in the presence of AP20187 and resulting binding to PI3P on early endosomes.

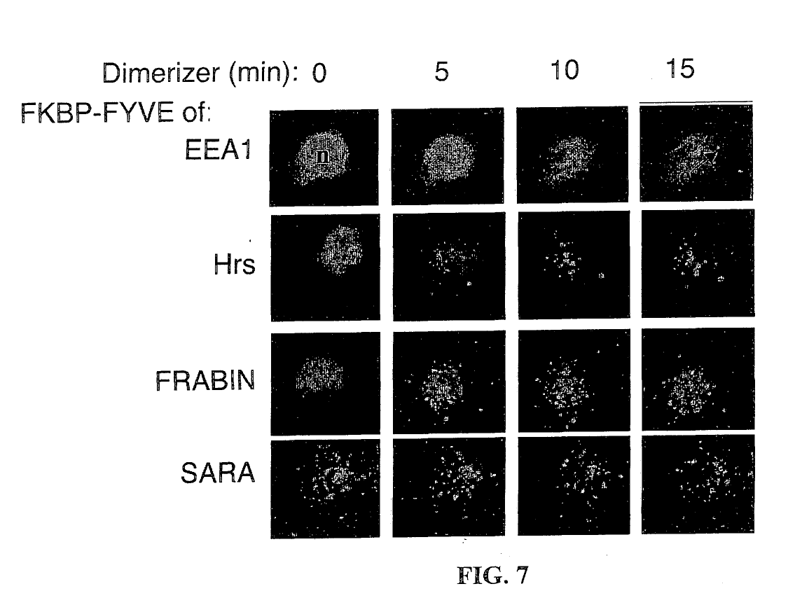

As seen in the chart in FIG. 7, punctate staining, evidence of endosome binding, is seen to result from these artificially dimerized lipid binding molecules. They varied, though, in their efficiency of binding. SARA has the greatest efficiency with almost immediate endosome binding upon addition of the chemical dimerizer, AP20187 (all four microscopic images show punctate staining), while EEA1 has the least efficiency with scarce punctate staining even 15 minutes after addition of the chemical dimerizer.

Based on the characteristics of a lipid binding domain displaying maximal affinity for lipid bilayers, as described above, a synthetic version of the lipid binding domain can be produced. An artificial FYVE domain would have a combination of high hydrophobicity, thus enabling hydrophobic interactions with bilayer lipids, and a strong positive charge to counteract the negative surface charge of the phospholipid bilayer. As noted above, examples of useful Turret loops include the amino acid sequences, AFFR (SEQ ID NO:14), AFIR (SEQ ID NO:15), AIFR (SEQ ID NO:16), AFFK (SEQ ID NO:17), AFIK (SEQ ID NO:18), AIFK (SEQ ID NO:19), and TFTK (SEQ ID NO:20). Lipid binding domains including these sequence should have a greater affinity than molecules including the naturally occurring SARA FYVE Turret loop.

The polypeptide sequence of the lipid binding domain can be prepared using naturally occurring or synthetic amino acids. In addition, peptidomimetics and peptoids can be artificially introduced to produce a synthetic, artificial lipid binding domain.

The crystal structure of the EEA1 protein can provide a model for use with currently available computer modeling programs which in conjunction with alignment of the critical amino acids found in the SARA FYVE domain described herein is useful in producing synthetic lipid binding domains.

Synthetic Amino Acids

Expression of a recombinant FYVE domain or purification of a protein, e.g., SARA protein, containing a FYVE domain in E. Coli or in insect cells are a common means of producing large quantities of the protein. However, these systems are sometimes limited in producing a correctly folded protein, for example. Utilizing the aforementioned critical amino acids, a synthetic lipid binding domain can be constructed. In one example, standard automated protein chemistry techniques can produce a peptide given an intended sequence. In another example, amino acid analogs such as alpha amino acids with substituted side chains, beta amino acids, reverse beta amino acids, bicyclo amino acid analogues, amino aldehydes, amino alcohols, N-alkylated amino acids, chiral diamines, diacids, beta amino aldehydes and amino sugars can be used and would have the advantage of being more stable to proteolysis, and thus would survive ingestion by a subject in the case where an oral formulation is administered. Custom synthesis is available from synthetic chemistry or medicinal chemistry companies such as Amino Acid Analogues, Inc. of Hopkinton, Mass.

With synthetic amino acids, one can make a new lipid binding domain. To test that a suitable lipid binding domain has been constructed, computer modeling programs can be used to analyze the new lipid binding molecule. Examples of such programs include the following: GRID (Goodford (1985) J. Med. Chem., 28:849-857); MCSS (Miranker and Karplus (1991) Proteins: Structure, Function and Genetics, 11:29-34); AUTODOCK (Goodsell and Olsen (1990) Proteins: Structure, Function, and Genetics, 8:195-202); and DOCK (Kuntz et al. (1982) J. Mol. Biol., 161:269-288). In addition, specific computer programs are also available to evaluate specific protein-protein interactions and the deformation energies and electrostatic interactions resulting therefrom. MODELLER is a computer program often used for homology or comparative modeling of the three-dimensional structures of proteins. See, Sali et al. (1993) J. Mol. Biol., 234: 779-815. A preselected polypeptide sequence to be modeled is aligned with one or more lipid binding domains whose crystal structures are known and the MODELLER program is used to calculate a full-atom model, based on optimum satisfaction of spatial restraints. Such restraints can include, inter alia, homologous structures, fluorescence spectroscopy, NMR experiments, or atom-atom potentials of mean force.

The Turret loop, and the WxxD (SEQ ID NO:11), (R/K)(R/K)HHCR (SEQ ID NO:12), and RCV motifs must be located at a suitable distance from each other in the lipid binding domain. For example, based on the crystal structure of EEA1, the W of the WxxD motif should be spaced about 3.5 Angstroms from the V of the RVC motif and 11 angstroms from the Turret loop when the lipid binding domain is properly folded. Custom synthesizing companies can tailor the product such that these distances are incorporated.

Peptidomimetics

Chemical variants of the lipid binding domains can be produced. For example, the side chains of the basic polypeptide lipid binding domain should be substantially maintained, but the chemical backbone of the polypeptide can be substituted, compared with the naturally occurring FYVE domain, at one or more peptide bonds to produce a peptidomimetic lipid binding domain. Such peptidomimetics can be produced by solid-phase synthesis. Companies such as Pepscan Systems (Netherlands) and Genzyme Pharmaceuticals (Cambridge, Mass.) are capable of translating a peptide sequence into a peptidomimetic sequence.

Peptoids

Another way of producing an artificial polypeptide lipid binding domain is to synthesize a peptoid lipid binding domain. A peptoid is an oligomer of N-substituted glycines, and can be used in making an optimal lipid binding domain. A peptoid can be synthesized from a variety of different N-alkylglycines that have side chains similar to amino acid side chains, e.g., as described in Simon et al., (1992) PNAS 89:9367-9371. It can serve as a motif for the generation of chemically diverse libraries of novel molecules, for example. As an alternative to natural polymers, the peptoid lipid binding domains provide a modular system that allows one to synthesize monomers in large amounts. The monomers have a wide variety of functional groups presented as side chains from the oligomeric backbone, the linking chemistry is high yielding and amenable to automation. The linkage in a peptoid is resistant to hydrolytic enzymes such as proteases. Another advantage is that the monomers are achiral. Examples of peptoids and how they are synthesized are fully described in Simon et al., (1992) Proc. Natl. Acad. Sci., 89:9367-9371.

As a specific example, the peptide Gly-Leu-Ser-Ala-Leu (SEQ ID NO:21) would be designated Gly-Nleu-Nser-Nala-Nleu in the peptoid form in which N stands for N-substituted. A peptoid version of the new ligand binding domain, containing the optimal characteristics found in the synthetic lipid binding domain of SEQ ID NO:1 (or the SARA FYVE domain), both described herein, can benefit from these advantages and provide a more stable lipid binding molecule.

Reporter Groups

The reporter group of the lipid binding molecule can be tailored to serve at least two main purposes. First, the reporter group can be used to quantitate the level of PI3P produced by the enzyme phosphoinositide 3-kinase, e.g. PI3P in a sample, e.g., cell lysate. By detecting the level of its product, the activity level of PI3K can also be detected. Second, it may be used for in vivo or in vitro visualization of PI3P-containing intracellular organelles such as endosomes or PI3P-containing lipid bilayers such as liposomes.

In the first case, the reporter group of the lipid binding molecule can be bound to a ligand on a substrate for the purpose of separating and isolating molecules containing the reporter group from mixtures and solutions (e.g., glutathione-S-transferase, and glutathione (see, e.g., U.S. Pat. No. 5,654,176) or anti-glutathione-S-transferase antibody; hexa-histidine and Ni²⁺ (see, e.g., German Patent No. DE 19507 166); chitin binding protein and chitin; cellulase (CBD) and cellulose; maltose binding protein and amylose, or maltose; dihydrofolate reductases and methotrexate; FKBP and FK506, and an antibody and a peptide epitope (see, e.g., Kolodziej and Young (1991) Methods Enz. 194:508-519 for general methods of providing an epitope tag).

In the second case, the reporter group allows detection of lipid binding molecules containing the reporter group, which is visible by microscopy. The reporter group can be a fluorescent label, such as green fluorescent protein (GFP), or other luminescent proteins, or can be bound to a label, e.g., a fluorescently labeled antibody. Detection can be in tissue slices mounted on a slide (in vivo) or in cells cultured in a dish (in vitro).

Visualizing endosomes by microscopy can rely on the use of GFP or other fluorescent reagents as the reporter group of the lipid binding molecule. Screening of cells treated with dyes and fluorescent reagents is well known in the art. There is a considerable body of literature related to genetic engineering of cells to produce fluorescent proteins, such as modified GFP, as a reporter molecule. Some properties of wild-type GFP are disclosed by Morise et al. ((1974) Biochemistry 13:2656-2662), and Ward et al. ((1980) Photochem. Photobiol. 31:611-615). The GFP of the jellyfish Aequorea victoria has an excitation maximum at 395 nm and an emission maximum at 510 nm, and does not require an exogenous factor for fluorescence activity.

Numerous references describe the use of GFP proteins in biological systems. For example, WO 96/09598 describes a system for isolating cells of interest utilizing the expression of a GFP-like protein. WO 96/27675 describes the expression of GFP in plants. WO 95/21191 describes modified GFP protein expressed in transformed organisms to detect mutagenesis. U.S. Pat. Nos. 5,401,629 and 5,436,128 describe assays and compositions for detecting and evaluating the intracellular transduction of an extracellular signal using recombinant cells that express cell surface receptors and contain reporter gene constructs that include transcriptional regulatory elements that are responsive to the activity of cell surface receptors.

The new reporter groups can be prepared using standard techniques. In one example, a cDNA that expresses the GFP protein is fused in frame to the rest of the lipid binding molecule, for example by ligating the cDNA or the rest of the lipid binding molecule into a commercially available GFP-containing vector. The vector is then transfected into and amplified by bacteria that express the complete lipid binding molecule. Finally, the molecule can be isolated from the bacteria and purified for use.

Another way of making the GFP reporter is to have cells express this protein, and then linking the GFP to a separately prepared lipid binding domain. By standard techniques, a DNA construct much like that described in the above paragraph, differing in the promoter used is transfected into cells of interest. The promoter is selected according to the cell type. After allowing the cellular machinery to translate the protein, visualization by microscopy can be performed.

Preparation of Lipid Binding Molecules

The new lipid binding molecules can be made synthetically in parts, which are then linked together, they can be synthesized as whole molecules, or they can be produced in cultured cells, such as bacteria, as two separate parts that are later linked, or as whole molecules.

For example, in some embodiments, the sequence encoding the lipid binding domain can be cloned in frame into any commercially available vector containing a sequence encoding a reporter group, such as glutathione-S-transferase (GST). The resulting fusion protein (e.g., GST-SARA FYVE) can be produced in any cultured cells, such as bacterial cells, e.g., E. Coli, or insect cells, and purified using conventional techniques.

In other embodiments, the lipid binding domain and the reporter group are produced separately, for example by standard techniques such as cloning the nucleic acids in frame into separate vectors, culturing transfected cells, e.g., bacteria or insect cells, with the cloned nucleic acid construct, and purifying the individual proteins. The two proteins, the ligand binding domain and the reporter group, are then linked together covalently or non-covalently. For the purposes of in vivo detection of endosomes, covalent attachment is ideal. The ligand binding domain and the reporter group can be directly linked together by a peptide bond, synthesized as a single contiguous peptide as described in the above paragraph, or joined by a linking or spacer molecule. Particularly useful linking molecules are water-soluble, well-tolerated by biological systems, and flexible, e.g., polyethylene glycol-based linkers. Another useful linking molecule is APDP (N-[4-(p-azido-salicylamido)butyl]-3′-(2′-pyridyldithio) propionamido; Pierce), which is a photoreactive crosslinker that can be radiolabeled.

Dimerization

The dimerization of lipid binding domain monomers has been found to be important for binding to PI3P in the context of a lipid bilayer. One can incorporate a naturally occurring dimerization domain or a dimerization domain can be synthesized, for example, by incorporating a binder to a chemical dimerizer such as dimethyl adipimidate•2 HCl (DMA), dimethyl suberimidate•2 HCl (DMS), disuccinimidyl glutarate (DSG), disuccinimidyl suberate (DSS), dithiobis[succinimidylpropionate] (DSP), ethylene glycol bis[succinimidylsuccinate] (EGS), and Sulfo-DSP (a water-soluble form of DSP). Many homobifunctional and heterobifunctional chemical dimerizers are available from Pierce Biotechnology.

One way to make a lipid binding molecule dimer is to make a chimera of a lipid binding domain and reporter group plus a dimerization domain of a known dimerizing protein, e.g., any know member of protein homodimer families, including, but not restricted to, alkaline phosphatase, enolase, glutathione S-transferase, copper-zinc superoxide dismutase, Streptomyces subtilisin inhibitor, and triose phosphate isomerase. The chimera can be made using methods known in the art, basically linking them by producing them on a single polypeptide, e.g., ligating the nucleic acid of different members of the chimera such that the translated protein is in frame for all members, and expression is driven by a promoter functional in the system used for protein expression, transfecting the nucleic acid construct into the system, e.g., bacteria or insect cells, and once the system expresses protein, isolating and purifying the protein.

As seen in FIGS. 6A-C, artificially dimerizing FYVE domains fused to a mutated form of FKBP called FKBPF36V (phenylalanine replaces valine at amino acid position 36 of FKBP), which binds to the chemical crosslinker AP20187. FIG. 6A illustrates the fusion protein followed by FIG. 6B, which shows the structure of AP20187. The schematic in FIG. 6C illustrates a model of how this chemical dimerization method works at the cellular level. Briefly, GFP-FKBPF36V-FYVE monomers are in solution. Addition of AP20187 to the solution crosslinks two monomers to form a dimer. The dimer can then more readily bind to PI3P present in the lipid bilayer of the endosome as is seen by the microscopic images of FIG. 7.

The images in FIG. 7 shows microscope images of cells expressing one of four different FKBP-tagged FYVE domains, that of EEA1, that of Hrs, that of FRABIN, and that of SARA, at different time points after the addition of the chemical dimerizer AP20187, no dimerizer, 5 minutes, 10 minutes, and 15 minutes after addition of dimerizer. The bottom row shows that the SARA FYVE domain was able to bind to endosomes even before addition of the dimerizer, while FRABIN and Hrs were less efficient at doing so, and EEA1 was the slowest, binding only by 15 minutes after dimerizer addition.

These images show that the SARA FYVE domain has a superior ability to bind to endosomes in an efficient manner, again emphasizing that the non-conserved residues present in SARA FYVE are important in conferring its efficiency in lipid binding. These results also suggest that the natural ability of the SARA FYVE domain to dimerize, as seen by its ability to bind endosomes very well even before the addition of dimerizer, plays a key role in allowing better lipid binding. Thus, key residues found in the SARA FYVE domain are useful in modeling the new lipid binding domain.

Other chemical crosslinkers can be found commercially (hetero-bifunctional and homo-bifunctional chemical crosslinkers, Pierce Biotechnology Rockford, Ill.). By screening a peptide library for binders of a chosen chemical crosslinker, one of skill in this field can formulate an artificial dimerizer which functions similarly to FKBPF36V/AP20187.

Uses of the Lipid Binding Molecules

The new lipid binding molecules have many uses both in a research setting and clinically. The examples that follow illustrate this range of uses, from the molecular level, where products of an enzyme are detected as a measure of enzyme activity level, to detection of lipids at the cellular level, and finally to detection or screening for treatments applicable to an organism.

Methods of detecting lipids without the use of radioactivity is useful in providing a safer, more cost effective means of studying intracellular events such as endosomal trafficking and fusion as well as monitoring PI3-kinase activity. However, the use of radioactivity is not prevented by the use of this invention and in cases where radioactivity is appropriate, standard protocols are still applicable and radioactive moieties can be used as the reported group.

One application for the new lipid binding molecules is as screening agents to identify potential pharmaceutical compounds that are useful to treat infections by microorganisms that disrupt the endosomal system, such as bacteria (e.g., Salmonella) and mycobacteria (e.g., Mycobacterium tuberculosis). Infection of a phagosome, which is a membrane-bound vesicle formed in a cell by an inward folding of the cell membrane to hold foreign matter taken into the cell by phagocytosis, by, e.g., Mycobacterium tuberculosis, renders the phagosome incapable of fusing to a lysosome, which under normal conditions could digest, and thus inactivate, the foreign organism. By rendering the phagosome incapable of fusing, the Mycobacterium tuberculosis ensures its survival within the cell. The lipid binding molecules provide a tool for testing whether a test compound enables the phagosome to fuse to the lysosome.

The lipid binding molecules can also be used to study endosome fusion in real time, for example to diagnose Mycobacterium tuberculosis infections. Studies have reported that M. tuberculosis resides in the phagosome, a type of endosome, of the cell it has infected and there it prevents the fusion of the phagosome to the lysosome. Fusion to the lysosome is part of the phagosome maturation process that allows the degradation of foreign material. Because the phagosome is unable to fuse to the lysosome, it is unable to rid the cell of M. tuberculosis (see, e.g., Gillooly et al., (2001) J. Cell Biology, 155:15-17, and Fratti et al., (2001) J. Cell Biology, 154:631-644). Visualization of the phagosome with the use of a ligand binding molecule can thus serve to diagnose M. tuberculosis infection in a cell. This same process can be used to screen for potential pharmaceutical compounds that may be used as treatment for M. tuberculosis infection.

After contacting the phagosome with the test compound, visualization can help determine whether the compound is able to promote phagosome fusion to the lysosome. High magnification visualization of the cells would reveal the regained ability of the phagosome within the cell to fuse to the lysosome in the presence of a successful test compound and not in the absence of the test compound.

Visualization of intracellular organelles can be performed on either fixed cells, using fluorescently labeled antibodies, biological ligands, and/or nucleic acid hybridization probes, or live cells using multicolor fluorescent indicators and “biosensors.” The choice of fixed or live cells for visualization depends on the specific cell-based assay required.

Fixed cell assays are the simplest, since an array of initially living cells in a microtiter plate format can be treated with various compounds and doses being tested, then the cells can be fixed, labeled with specific reagents, and measured. No environmental control of the cells is required after fixation. Spatial information is acquired, but only at one time point. The availability of thousands of antibodies, ligands, and nucleic acid hybridization probes that can be applied to cells makes this an attractive approach for many types of cell-based screens. The fixation and labeling steps can be automated, allowing efficient processing of assays.

Live cell assays are more sophisticated and powerful, since an array of living cells containing the desired reagents can be screened over time, as well as space. Environmental control of the cells (temperature, humidity, and carbon dioxide) is required during measurement, since the physiological health of the cells must be maintained for multiple fluorescence measurements over time. There is a growing list of fluorescent physiological indicators and “biosensors” that can report changes in biochemical and molecular activities within cells (Giuliano et al., (1995) Ann. Rev. Biophys. Biomol. Struct. 24:405; Hahn et al., (1993) In Fluorescent and Luminescent Probes for Biological Activity. W. T. Mason, (ed.), pp. 349-359, Academic Press, San Diego).

The availability and use of fluorescence-based reagents has helped to advance the development of both fixed and live cell high content screening (HCS). Advances in instrumentation to automatically extract multicolor, high-content information have recently made it possible to develop HCS into an automated tool. An article by Taylor, et al. (American Scientist 80 (1992), p. 322-335) describes many of these methods and their applications. For example, Proffitt et. al. (Cytometry 24:204-213 (1996)) describe a semi-automated fluorescence digital imaging system for quantifying relative cell numbers in situ in a variety of tissue culture plate formats, especially 96-well microtiter plates. The system consists of an epifluorescence inverted microscope with a motorized stage, video camera, image intensifier, and a microcomputer with a PC-Vision digitizer. Turbo Pascal software controls the stage and scans the plate taking multiple images per well. The software calculates total fluorescence per well, provides for daily calibration, and configures easily for a variety of tissue culture plate formats. Thresholding of digital images and reagents that fluoresce only when taken up by living cells are used to reduce background fluorescence without removing excess fluorescent reagent. HCS for the tracking of membrane trafficking is described by Cellomics, Inc. who provides a kit for this use.

Scanning confocal microscope imaging (Go et al., (1997) Analytical Biochemistry 247:210-215; Goldman et al., (1995) Experimental Cell Research 221:311-319) and multiphoton microscope imaging (Denk et al., (1990) Science 248:73; Gratton et al., (1994) Proc. of the Microscopical Society of America, pp. 154-155) are also well established methods for acquiring high resolution images of microscopic samples. The principle advantage of these optical systems is the very shallow depth of focus, which allows features of limited axial extent to be resolved against the background. For example, it is possible to resolve internal cytoplasmic features of adherent cells from the features on the cell surface. Because scanning multiphoton imaging requires very short duration pulsed laser systems to achieve the high photon flux required, fluorescence lifetimes can also be measured in these systems (Lakowicz et al., (1992) Anal. Biochem. 202:316-330; Gerrittsen et al. (1997), J. of Fluorescence 7:11-15), providing additional capability for different detection modes. Small, reliable and relatively inexpensive laser systems, such as diode pumped lasers, are now available to allow multiphoton confocal microscopy to be applied in a fairly routine fashion.

Because the new lipid binding molecules bind to lipids such as PI3P with high specificity and affinity, they can also be used to target such lipids, e.g., to deliver a therapeutic agent, such as an antibiotic, to a lipid location within a cell or organism. In addition, they can be used to image such lipid locations by using reporter groups that can be detected and monitored in living cells and organisms, such as by x-ray, magnetic resonance imaging (MRI), or fluorescence. In other embodiments, by binding to endosomes with a high binding affinity, the new lipid binding molecules can inhibit the endosomal systems of specific microorganisms, such as fungi, if the lipid binding domain is created to specifically bind to the PI3P of the target microorganism. Thus, the lipid binding molecules have various therapeutic and diagnostic applications.

Pharmaceutical Compositions and Administration

For use in these various applications, the invention includes compositions, e.g., pharmaceutically acceptable compositions, which include the new lipid binding domains and molecules, either by themselves or linked to another molecule, e.g., an antibiotic or drug, such as an anti-tuberculosis drug, such that the ligand binding domain targets the drug to a lipid location, such as the endosome. The pharmaceutically acceptable compositions are typically formulated together with a pharmaceutically acceptable carrier.

Pharmaceutical compositions can be manufactured with a pharmaceutically acceptable carrier. Examples of pharmaceutical carriers include any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., ligand binding domain or molecule, may be coated in a material to protect the compound from the action of acids and other natural conditions that may inactivate the compound.

Some pharmaceutical compositions contain a pharmaceutically acceptable salt. This refers to a salt that retains the desired biological activity of the parent or active compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine, and the like.

The compositions of this invention may be in a variety of forms. These include, for example, liquid, semi-solid, and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical compositions are in the form of injectable or infusible solutions, such as compositions similar to those used for administration to humans or animals with small polypeptides or antibodies. A useful mode of administration is parenteral (e.g., intravenous, subcutaneous, intraperitoneal, intramuscular). In another embodiment, the lipid binding domain or molecule linked to a drug is administered by intramuscular or subcutaneous injection.

Modes of administering a pharmaceutical composition include parenteral administration, enteral administration, and topical administration. Parenteral administration is usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. A pharmaceutical composition can also be tested to insure it meets regulatory and industry standards for administration. For example, endotoxin levels in the preparation can be tested using the Limulus amebocyte lysate assay (e.g., using the kit from Bio Whittaker lot # 7L3790, sensitivity 0.125 EU/mL) according to the USP 24/NF 19 methods. Sterility of pharmaceutical compositions can be determined using thioglycollate medium according to the USP 24/NF 19 methods. For example, the preparation is used to inoculate the thioglycollate medium and incubated at 35° C. for 14 or more days. The medium is inspected periodically to detect growth of a microorganism.

The compositions can be formulated as a solution, microemulsion, dispersion, liposome, or other ordered structure suitable to high drug concentration. Sterile injectable solutions can be prepared by incorporating the active compound (i.e., the ligand) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The new ligand binding molecule compositions can be administered by a variety of methods known in the art, although for many applications, intravenous injection or infusion is a useful route of administration. For example, for therapeutic applications, the ligand binding molecules linked to a drug or antibiotic can be administered by intravenous infusion at a rate of less than about 30, 20, 10, 5, or 1 mg/min to reach a dose of about 1 to 100 mg/m² or 7 to 25 mg/m² of patient skin surface area. The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

In certain embodiments, the pharmaceutical composition may be orally administered, for example, with an inert diluent or an assimilable edible carrier. The synthetically produced version of the ligand binding molecules would be ideal in this scenario since amino acid analogs or artificial peptide bonds should better withstand digestion. The ligand binding domains or molecules (and other ingredients, if desired) can also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

In other embodiments, pharmaceutical compositions can be administered with medical devices known in the art. For example, in one embodiment, a pharmaceutical composition of the invention can be administered with a needle-less hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

In certain embodiments, the compounds of the invention can be formulated to ensure proper distribution in vivo. For example, the blood-brain barrier (BBB) excludes many highly hydrophilic compounds. To ensure that therapeutic compounds cross the BBB (if desired), they can be formulated, for example, in liposomes. For methods of manufacturing liposomes, see, e.g., U.S. Pat. Nos. 4,522,811; 5,374,548; and 5,399,331. The liposomes can comprise one or more moieties, which are selectively transported into specific cells or organs, thus enhance targeted drug delivery (see, e.g., V. V. Ranade (1989) J. Clin. Pharmacol., 29:685).

Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. “Dosage unit form” as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention is dictated by and directly dependent on (a) the unique characteristics of the active compound and the particular therapeutic effect to be achieved, and (b) the limitations inherent in the art of compounding such an active compound for the treatment of sensitivity in individuals.

For any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a therapeutically effective amount or a prophylactically effective, amount of a ligand binding molecule, e.g., linked to an antibiotic or other drug. A therapeutically effective amount refers to an amount effective, at dosages and for periods of time necessary, to achieve a desired therapeutic result. A therapeutically effective amount of the composition may vary according to factors such as the disease state, age, sex, and weight of the individual, and the ability of the protein ligand to elicit a desired response in the individual. A therapeutically effective amount is also one in which any toxic or detrimental effects of the composition is outweighed by the therapeutically beneficial effects.

A prophylactically effective amount refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

### EXAMPLES

The invention is further described in the following examples, which do not limit the scope of the invention described in the claims.

### Example 1

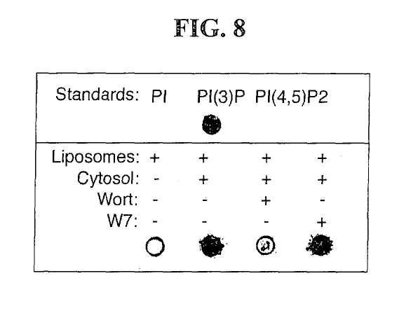

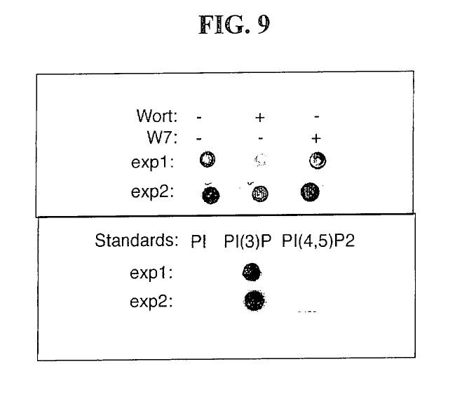

In this example, the lipid binding molecule is used to determine the amount of PI3K product in a sample, thus detecting the activity level of PI3K. The FYVE domain of SARA is fused to a reporter group such as glutathione S-transferase. FIGS. 8 and 9 illustrate the results of this example. In this example, postnuclear supernatants (Cytosol, cell lysate immunoprecipitates, or recombinantly expressed PI3K can be used as a source of enzyme) were prepared by scraping cells into 3 mL of cytosol buffer (25 mM HEPES, pH 7.0, 125 mM potassium acetate, 2.5 mM magnesium acetate, 0.2 M sucrose, 1 mM dithiothreitol, 1 mM ATP, 5 mM creatine phosphate, 0.01 mg/ml creatine phosphokinase with 10 μg/ml leupeptin, 1 mM TAME, 1 mM 1,10-phenantroline), homogenizing by repeated passage (7-10 times) through a 27-gauge needle and centrifugation at 1000×g for 5 minutes to remove nuclei and unbroken cells. Aliquots of the postnuclear extract (100 μl) were dispensed into three aliquots, which were incubated with no additions, 100 nM wortmannin (Wort), an inhibitor of PI3K, or 100 μM W7 for 10 minutes. W7 is a calmodulin inhibitor used here because the EEA1 protein is known to disrupt EEA1 binding. W7 does not inhibit PI3K activity and so adequately separates these processes.

Membranes were pelleted by high-speed centrifugation, and resuspended in cytosol buffer. Lipids were extracted by addition of chloroform:methanol. The organic phase was removed, dried, resuspended by sonication in PBS and spotted onto Hydrobond® nitrocellulose membranes using a dot-blotting manifold. The nitrocellulose blot was then probed with 2.5 μg/ml of a GST-fusion protein of the FYVE domain of SARA. After overnight incubation at 5° C., blots were washed and probed with anti-GST antibody. Bound antibody was detected using horseradish peroxidase-conjugated goat anti-mouse secondary antibody (Promega), which was detected by Renaissance enhanced luminol reagent (PerkinElmer Life Sciences).

As seen in FIG. 8, the cytosol of the extract, which contains PI3 kinase activity, is incubated with liposomes containing PI. The levels of PI 3kinase are then assessed by measuring the levels of PI3P formed, which appear as the dots at the bottom. In the absence of liposomes, there is no signal, as expected, because the PI3P formed by the enzyme resides in the liposomes. In the presence of an inhibitor of PI3K, wortmannin, there is no signal, because the enzyme was unable to phosphorylate phosphatidylinositol and so could not produce PI3P. The absence of a signal when cytosol is absent is also expected, because the PI3K activity in this experiment resides in the cytosol.

FIG. 9, which shows the results of two experiments in which the assay is used to detect levels of PI3P in the membranes obtained from the cells. In these experiments, lipids are extracted from biological membranes from live cells, and the whole unfractionated lipid extract is spotted. This is in contrast to the experiment shown in FIG. 8, where the lipids were simple mixtures of PI (phosphatidylinositol) and PS (phosphatidylserine) that were exposed to a source of PI3 kinase. The complexity of the lipid extract from cellular membranes requires that a probe for PI3P have extremely high affinity and specificity. The ability of the lipid binding molecule composed of GST fused to the FYVE domain of SARA to detect PI3P in these complex lipid mixtures and thus PI3K activity, further illustrates the utility of the new lipid binding molecules. These results indicate that the lipid binding molecule of GST fused to the FYVE domain of SARA allows the quantitative detection of PI3P product, and thus the level of activity of PI3K in intact cells as read by GST detected by the anti-GST antibody via chemiluminescence (by standard procedures).

### Example 2

In another example, two monomers of the SARA FYVE domain that are fused to FKBPF36V (a mutated form of FKBP) were dimerized using the chemical dimerizer AP20187, which binds to this mutated form of FKBP. This lipid binding molecule has GFP as its reporter group. The lipid binding molecule was then transfected into COS7 cells, by calcium phosphate precipitation, for the purpose of visualizing endosomes by microscopy. After 24 hours, the cells were imaged using high speed, three-dimensional microscopy. Dimerizer AP20187 was used at a final concentration of 100 nM. The microscope was configured to 333 nm/pixels using a 60 Å objective, and the laser illumination was configured to provide a 488 nm excitation wavelength with a flux on specimen of ˜12 watts/cm². Exposure times of 5 ms were used to acquire each of 18 optical sections, spaced by 250 nm. Each set of 18 optical sections was acquired in less than 1 second, allowing 20 ms for each 250-nm shift in focus. Stacks were acquired every 10 seconds for 20 continuous minutes. Stacks were projected into single two-dimensional images.

As seen in FIG. 7, endosomes are readily visible by this method. FIG. 7 provides images of punctate staining, which indicates binding of the lipid binding molecule to the endosome at certain times after the addition of the chemical dimerizer. As seen in FIG. 7, the lipid binding molecule that includes the SARA FYVE domain is most efficient at binding to endosomes, seeming to bind equally in the presence or absence of dimerizer. This ability of SARA FYVE domain to bind is due to the composition of its turret loop, as well as to the composition of its dimer interphase, which can induce the formation of FYVE domain dimers without additional exogenous dimerizers.

### Example 3

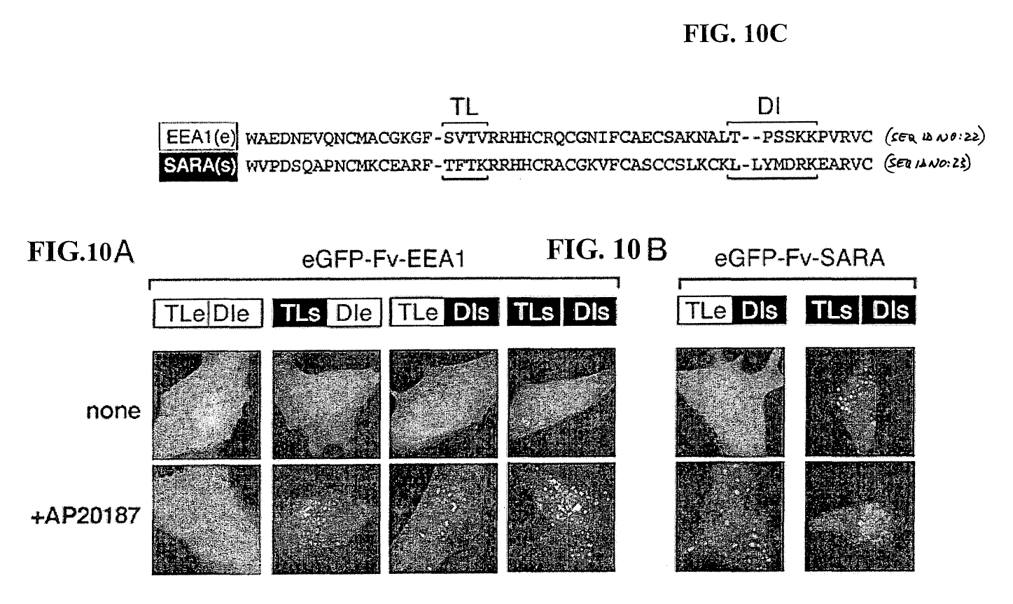

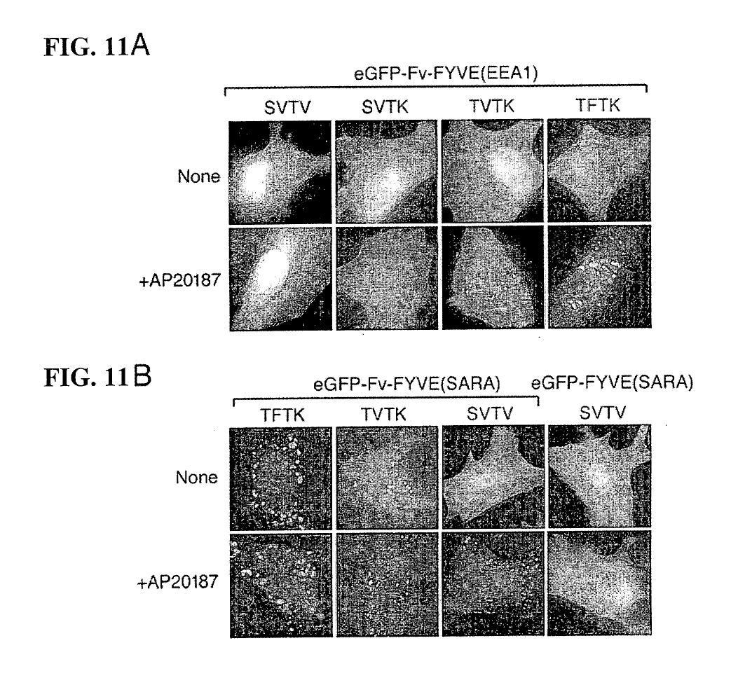

FIGS. 10A and 10B illustrate the subcellular localization of chimeras containing diverse combinations of four amino acids of the turret loops (TL) and six amino acids of the dimer interface loops (DI) of EEA1 and SARA. As shown, eGFP-Fv-FYVE(EEA1) displayed a cytosolic distribution both in the absence and presence of dimerizer (FIG. 10A, TLeDIe). However, when four amino acids in the turret loop of EEA1 were replaced by those of SARA, strong binding to endosomes was observed in response to dimerizer (FIG. 10A, TLs/DIe). These results suggest that the turret loop of SARA contributes significantly to the membrane partitioning of the SARA FYVE domain. Consistent with this hypothesis, replacement of the corresponding amino acids in eGFP-Fv-FYVE(SARA) with those of EEA1 caused the protein to shift from its characteristic endosomal localization (FIG. 10B TLs/DIs) to a cytoplasmic distribution (FIG. 10B, TLe/DIs), and to require the addition of dimerizer to regain an endosomal steady-state localization.

Interestingly, replacement of six amino acids in the region of the dimer interface of EEA1 with the corresponding region of SARA also resulted in significant endosomal binding in response to dimerization (FIG. 10A, TLe/D Is). When both the turret loop and the dimer interface region of the FYVE domain of EEA1 were replaced with those of SARA, the resulting construct displayed detectable endosome binding even in the absence of dimerizer, and very strong binding in its presence (FIG. 10A, Tls/DIs). This phenotype approximates that of the FYVE domain of SARA, which localizes to endosomes in the absence or presence of dimerizer (FIG. 10B, Tls/DIs). These results indicate that the extreme difference in the ability of the SARA and EEA1 FYVE domains to localize to endosomes is due to differences in the structure of both the turret loop and dimer interface regions. These regions are illustrated in FIG. 10C, which shows the relevant FYVE domain (up to the RVC motif) of EEA1 (SEQ ID NO:22) and SARA (SEQ ID NO:23).

### Example 4

FIGS. 11A and B illustrate the effect of hydrophobic and electrostatic interactions on the ability of the turret loop to induce endosomal localization of FYVE domains. Four amino acids in the turret loop of EEA1 were replaced one by one with those of SARA As shown in FIG. 11A, substitution of the second valine of the EEA1 turret loop with a lysine caused a significant enhancement in endosomal targeting (SVTK), evidenced by the finding of detectable endosomal labeling in response to dimerizer. Replacement of the first serine with threonine (TVTK) had no further significant effect, but replacement of the first valine with phenylalanine greatly enhanced endosomal binding (TFTK).

These results indicate that addition of a positive charge as well as of a more hydrophobic moiety significantly enhances the avidity of FYVE domains for endosomes. Consistent with this hypothesis, replacement of the phenylalanine residue in the turret loop of the SARA FYVE domain with a valine (TVTK) resulted in significant displacement from the endosomal localization observed at steady-state (FIG. 11B), but a more significant effect was observed upon replacement of the positively charged lysine with valine (SVTV). Thus, both positive charge and hydrophobicity provide the structural requirements for the enhancement of endosome binding attributable to the turret loop. These results can be used to prepare artificial lipid binding domains that bind to lipids with high affinity.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the following claims.

What is claimed is:

1. An isolated lipid binding domain comprising the sequence
(SEQ ID NO:1)
WxxDxxxxxCxxCxxxx(A/T)uuj(R/K)(R/K)HHCR(A/G/V)Cxxx
xCxxCxxxxxxxxxxuuuxuuRVCxxCx,
wherein x is any amino acid; u is a highly hydrophobic residue; and j is a positively charged residue.

2. The lipid binding domain of claim 1, wherein u is F, V, I, L, M, or W.

3. The lipid binding domain of claim 1, wherein j is R or K.

4. The lipid binding domain of claim 1, wherein each x is selected to be the same as a corresponding amino acid in SARA protein.

5. The lipid binding domain of claim 1, where the sequence comprises the FYVE sequence of the SARA protein (SEQ ID NO:10).

6. The lipid binding domain of claim 1, wherein the domain comprises a synthetic sequence.

7. The lipid binding domain of claim 1, where the domain comprises a peptoid sequence.

8. The lipid binding domain of claim 1, where the domain comprises a peptidomimetic sequence.

9. The lipid binding domain of claim 1, where the domain comprises a Turret loop selected from the group consisting of: AFFR (SEQ ID NO:14), AFIR (SEQ ID NO:15), AIFR (SEQ ID NO:16), AFFK (SEQ ID NO:17), AFIK (SEQ ID NO:18), AIFK (SEQ ID NO:19), and TFTK (SEQ ID NO:20).

10. The lipid binding domain of claim 1, wherein u is Y, M, T, or F.

11. A lipid binding molecule comprising
(a) a lipid binding domain of claim 1; and
(b) a reporter group linked to the lipid binding domain.

12. The lipid binding molecule of claim 11, wherein the lipid binding domain binds to phosphatidylinositol 3-phosphate.

13. The lipid binding molecule of claim 11, wherein the lipid binding domain binds to phosphatidylinositol 3-phosphate on an endosome.

14. The lipid binding molecule of claim 11, wherein the lipid binding domain binds to phosphatidylinositol 3-phosphate on a liposome.

15. The lipid binding molecule of claim 11, wherein the reporter group binds to a substrate.

16. The lipid binding molecule of claim 11, wherein the reporter group comprises one member of a binding pair, and wherein a substrate comprises a second member of the binding pair.

17. The lipid binding molecule of claim 11, wherein the reporter group is selected from the group consisting of glutathione-S-transferase (GST), His X6 (6His), FLAG, Green Fluorescent Protein, chitin binding protein, cellulase, maltose binding protein, dihydrofolate reductases, FK506 binding protein (FKBP), FKBPF36V, an antibody, a fluorescently labeled antibody, and an antibody fragment.

18. The lipid binding molecule of claim 11, wherein the reporter group comprises a fluorescent moiety.

19. The lipid binding molecule of claim 18, wherein the fluorescent moiety comprises GFP.

20. A method for detecting a lipid within a lipid bilayer in a sample, the method comprising:
a) obtaining a lipid binding molecule of claim 11;
b) mixing the lipid binding molecule with the sample under conditions that enable the lipid binding molecule to bind to a lipid in the sample to form a complex; and
c) detecting the complex as an indication of the presence of the lipid in the sample.

21. The method of claim 20, wherein the lipid comprises phosphatidylinositol 3-phosphate.

22. The method of claim 20, wherein the lipid binding domain comprises a SARA FYVE domain.

23. The method of claim 20, wherein the lipid binding domain comprises naturally occurring amino acids.

24. The method of claim 20, wherein the lipid binding domain comprises synthetic amino acids.

25. The method of claim 20, wherein the reporter group comprises glutathione S-transferase.

26. The method of claim 20, wherein the lipid comprises a cellular lipid membrane.

27. The method of claim 26, wherein the cellular lipid membrane comprise an endosome.

28. A method of locating a lipid-containing cellular organelle within a cell, the method comprising
a) obtaining a lipid binding molecule of claim 11;
b) applying the lipid binding molecule to the cell under conditions that enable the lipid binding molecule to enter the cell; and
c) detecting the reporter group of the lipid binding molecule, whereby the location of the reporter group within the cell indicates the location of the cellular organelle.

29. The method of claim 28, wherein the method is performed in vivo.

30. The method of claim 28, wherein the method is performed in vitro.

31. The method of claim 28, wherein the cellular organelle comprises phosphatidylinositol 3-phosphate.

32. The method of claim 28, wherein the cellular organelle is an endosome.

33. The method of claim 32, wherein the endosome comprises a phagosome.

34. The method of claim 28, wherein the cellular organelle is a liposome.

35. The method of claim 28, wherein the reporter group provides a visual signal and the method further comprises visualizing the location of the cellular organelle.

36. A method of diagnosing a subject for infection by a microorganism, the method comprising
a) obtaining a cell from the subject;
b) binding a lipid binding molecule of claim 11 to a phagosome in the cell;
c) visualizing the reporter group on a phagosome; and
d) determining whether the phagosome is capable of fusing to a lysosome, wherein a phagosome that cannot fuse to a lysosome indicates that the cell is infected by a microorganism.

37. The method of claim 36, wherein the microorganism is Mycobacterium tuberculosis.

38. The method of claim 36, wherein the cell is derived from a subject at risk for infection by the microorganism.

39. The method of claim 36, wherein the subject is a mammal.

40. The method of claim 36, wherein the subject is a human.

41. A method of determining whether a test compound is a candidate compound for treating Mycobacterium tuberculosis, the method comprising
a) binding a lipid binding molecule of claim 11 to a phagosome in a cell infected by M. tuberculosis;
b) applying a test compound to the infected cell; and
c) visualizing the phagosome before and after application of the test compound;
wherein a phagosome that is capable of fusing to a lysosome after application of the test compound indicates that the test compound is a candidate compound to treat M. tuberculosis.

42. The method of claim 41, wherein the infected cell is a cultured cell.

43. The method of claim 41, wherein the infected cell is derived from a subject.

44. The method of claim 41, wherein the subject is a mammal.

45. The method of claim 41, wherein the subject is a human.

46. The method of claim 41, wherein the reporter group is visible by microscopy.

47. The method of claim 41, wherein the reporter group comprises a fluorescent compound.
